(19) United States
(12) Patent Application Publication
(10) Pub. No.: US 2008/0097278 A1
(43) Pub. Date: Apr. 24, 2008

(54) Treatment of Skin with Light and a Benefit Agent

(76) Inventors: Curtis A. Cole, Ringoes, NJ (US); Elvin Lukenbach, Flemington, NJ (US); Gregory Skover, Princeton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/572,166

(22) PCT Filed: Jul. 15, 2005
(86) PCT No.: PCT/US05/25143

§ 371 Date: Jan. 16, 2007

Related U.S. Application Data
(60) Provisional application No. 60/588,715, filed on Jul. 16, 2004.

(51) Int. Cl. A61N 5/06; A61K 35/00; A61K 38/02

(52) U.S. Cl. 604/20; 132/286; 514/2; 514/789; 606/9

(57) ABSTRACT

Methods of mitigating effects of aging on skin include a first skin treatment to an expanse of skin, and after a delay, providing a second skin treatment to the same expanse of skin. The skin treatments may include initiating exposure of an expanse of skin to light; terminating the exposure of the expanse of skin to the light after a period, preferably of less than about one hour; and applying a first benefit agent treatment to the expanse of skin after a first delay following the termination.

### FIELD OF THE INVENTION

The present invention relates to treatment of the skin and, more particularly, to the application of light to the skin, followed by the topical application of a benefit agent to said skin.

### BACKGROUND OF THE INVENTION

With advances in nutrition and medical treatment, the life expectancy of the average U.S. and world citizen has increased dramatically. As a result, large portions of those populations suffer from the associated effects of aging, including an increasing number of skin health issues. Though seldom life threatening, skin health issues can be uncomfortable and may cause chronic disabilities. In addition, because the skin is so visible, skin health issues and cosmetic skin conditions can lead to psychological stress in the patients who have them. Many members of the aging population have also become increasingly educated regarding general physical health and ways of looking and feeling better about physical appearance. This desire for good health and appearance has driven people to seek improved solutions to health care and skin care.

Numerous techniques have been proposed to provide cosmetic and/or anti-aging or skin rejuvenation benefits. For example, it has been proposed to expose the skin to electromagnetic radiation. The electromagnetic radiation typically includes wavelengths that are absorbed by at least one chromophore present in the skin, (e.g. melanin, hemoglobin) such that the incident energy can be converted to heat. If sufficient energy is delivered and absorbed, one or more benefits such as age spot reduction, mottled hyperpigmentation reduction, wrinkle reduction, blood vasculature reduction, reduction of skin roughness, and lifting of sagging skin may be imparted to the skin.

One example of a device offered by professional dermatologists that emits electromagnetic energy to deliver skin benefits (hair removal as well as the above-mentioned anti-aging benefits) is the xenon “flashlamp”. Flashlamps are useful as therapeutic light sources since they are able to provide illumination across a large area of the skin at any given time. Typical flashlamps are broadband energy sources that emit non-coherent electromagnetic radiation in across the visible spectrum (as well as parts of the infrared spectrum). The light source is typically delivered in pulses having duration on the order of a millisecond.

Other electromagnetic devices used to treat skin include those that deliver electromagnetic radiation in narrow “beams” such as those from lasers and light emitting diodes. Such electromagnetic radiation may be focused to a small spot size to enable treatment of specific regions of the skin. Alternatively, such devices may be progressively repositioned (stamped) across a large area of the skin to deliver certain benefits.

Topical treatments are also used to treat various indications of skin aging. For example, topical application of vitamins such as vitamin A and its derivatives is known to be an effective treatment for wrinkles and other signs of skin aging. Furthermore, it is known to use topical compositions with light for treating the skin. For example, O'Donnell (U.S. Pat. No. 6,106,514) teaches a method for delivering pulsed infrared laser energy to increase skin tone. The laser energy has a fluence of greater than 100 J/cm2. A topical post-treatment with anti-inflammatory, anti-oxidant, and neo-collagen promoting substances may be applied to the treated area for 30to 90 days.

McDaniel (U.S20030004499 and W02003001894) teaches a method for dermatological treatment using narrowband, multichromatic electromagnetic radiation. A topical pre-treatment, such as an exogenous chromophore or a cosmaeceutical may be used to enhance the penetration of light. The procedure may be repeated every 1 to 60 days.

Korman (US20020128695A1) teaches a method for high energy photodynamic therapy of acne vulgaris and seborrhea. The method includes illuminating a skin area with narrow-band, high intensity light having spectral characteristics of at least one of a group of narrow spectral bands consisting of 400 nm-450 nm (blue), 520 nm-550 nm (green) or 630 nm-670 nm (red) spectral range. The light source generates a high intensity, non-coherent light in exact narrow spectral bands needed for activation of the photodynamic reaction while filtering out harmful UV light. Pre-treatment with oxygen transporting compounds, perfluorocarbons, oxidative substances such as a hydrogen peroxide compound, keratolytic substances and external photosensitizers such as methylene blue may be performed. The function of these pre-treatments is to release oxygen directly into the seabacious glands and raise the efficiency of the destruction of p. acnes.

Perricone (US20030009158A1) describes irradiating affected skin with blue and/or violet light. Compounds containing alpha hydroxyacids may be applied prior to phototreatment to increase light penetration into the skin.

Anderson (US20020099094) teaches light treatment of sebaceous gland disorders with 5-aminolevulinic acid (ALA) and photodynamic therapy. The ALA is described as metabolized via the porphyrin pathway. A metabolite infiltrates the skin to be treated. When intense light with a wavelength between 320 and 700 is delivered to ALA-treated skin, the excited metabolite (photoporphyrin IX) is excited and reacts with oxygen to produce singlet oxygen.

Tankovitch (U.S. Pat. No. 6,162,211A) teaches imbeddeding a material (e.g., graphite) that has a high optical absorbance at or near at least one frequency band of light that will penetrate the skin into the skin in order to absorb light energy and effect the surrounding tissue.

The preceding examples illustrate that conventional treatment of the skin using electromagnetic radiation employs a monotherapy approach. For example, in conventional treatment, the skin is exposed to electromagnetic radiation, perhaps after a chromophore is topically applied thereto. The radiation is absorbed by the skin and the chromophore, and heat is dissipated to the nearby tissue. As such, only a single biological pathway (thermal injury/recovery) is employed to affect a particular benefit. This is unfortunate, since this solitary mechanism is prone to diminishing returns as the fluence, frequency or time of radiation is increased. In many cases, saturation of the benefit is achieved beyond a certain frequency, fluence, or time of treatment.

Accordingly, conventional practices are subject to several drawbacks. Firstly, electromagnetic radiation having a high energy density (fluence) is often utilized. The high energy density delivered may be unsafe for a lay user (e.g., a consumer) to use in a home setting. Furthermore, high fluence radiation tends to heat the skin to an uncomfortable temperature and therefore require that the skin be cooled during operation. For example, for devices that contact the skin, this uncomfortable heating may require that a skin-cooling system be built into the device itself, which can be expensive or limiting to the device design.

For other conventional practices, the fluence of radiation is too low to deliver adequate efficacy. Even if the patient goes through the inconvenience and expense of making frequent visits to a professional skin care specialist to receives multiple treatments, the results are often unsatisfactory. Furthermore, treatment with electromagnetic radiation alone does not impart protection from further aging-related degradation of the treated tissue that may result in the future.

Therefore, a need exists for a system for treating the skin that overcomes one or more of the above-mentioned drawbacks.

### SUMMARY OF THE INVENTION

In one aspect, embodiments of the invention relate to a method of mitigating effects of aging on skin. In a first embodiment, the method includes providing a first skin treatment to an expanse of skin, and after a delay, providing a second skin treatment to the same expanse of skin. The first skin treatment includes initiating exposure of an expanse of skin to light; terminating the exposure of the expanse of skin to the light after a period, preferably of less than about one hour; and applying a first benefit agent treatment to the expanse of skin after a first delay following the termination. The light is primarily within about 400 nm to about 850 nm with a fluence of about 5 J/cm2 to about 100 J/cm2. The second skin treatment includes initiating exposure of an expanse of skin to light; terminating the exposure of the expanse of skin to the light after a period, preferably of less than about one hour; and applying a the first benefit agent treatment to the expanse of skin after a delay following the termination. This delay may be similar to the first delay, but the second delay is of greater duration that the first delay. At least one additional benefit agent treatment may also be applied during the second delay.

In another embodiment of the invention, a method of mitigating effects of aging on skin includes the steps of exposing an expanse of skin to light for a period, preferably of less than about one hour; terminating the exposure of the skin to the light; and applying a benefit agent to the expanse of skin after a delay lasting less than 12 hours following the termination. The light is primarily within about 400 nm to about 850 nm with a fluence of about 5 J/cm2 to about 100 J/cm2.

In another aspect of the invention, a method of promoting a topical composition, the method includes the steps of instructing a user to topically apply said composition to an expanse of skin following an exposure-of said expanse of skin to light. The light is substantially free of ultraviolet radiation; is primarily within about 400 nm to about 850 nm; and provides a fluence of about 5 J/cm2 to about 100 J/cm2, having selected wavelengths and/or wavelength bands, primarily within the spectral range of about 400 nm to about 850 nm wherein said light source delivered from about 0.01 Watt/cm2 to about 100 W/cm2 to the skin wherein the total fluence delivered is less than 100 J/cm2. Preferably, the light exposure is completed within 24 hours prior to said topical application.

In another aspect of the invention, a kit includes a light source, a benefit agent, and instructions. The light source provides a fluence of about 5 J/cm2 to about 100 J/cm2 of light primarily within about 400 nm to about 800 nm. The instructions relate to the application of at least one treatment of the benefit agent to the skin within 24 hours immediately following exposure of skin to light from said light source.

### BRIEF DESCRIPTION OF THE DRAWINGS

A more particular description of the invention, briefly summarized above may be had by reference to the embodiments thereof that are illustrated in the appended drawings. It is to be so noted, however, that the appended drawings illustrate only typical embodiments of the invention and, therefore, are not to be considered limiting of its scope, for the invention may admit to other equally effective embodiments.

FIG. 1 is a schematic side view of an expanse of skin being treated with light, according to embodiments of the invention described herein;

FIG. 2a is a schematic top view of an expanse of skin being treated with light;

FIG. 2b is a schematic top view of an expanse of skin, and light being progressively repositioned across the expanse of skin; and

FIG. 3 is a schematic side view of a device capable of being progressively repositioned across an expanse of skin in a manner consistent with embodiments of the invention described herein.

To facilitate understanding identical reference elements have been used, wherever possible, to designate identical elements that are common to the figures.

### DETAILED DESCRIPTION OF THE INVENTION

Unless defined otherwise, all technical and scientific terms used herein have the same meaning as commonly understood by one of ordinary skill in the art to which the invention belongs.

### Light Exposure

Embodiments of the invention includes apparatus and methods for mitigating the effects of aging on skin. By “mitigating the effects of aging on skin,” it is meant one or more of the following benefits are imparted to a subject's skin: skin rejuvenation benefits such as younger, healthier, radiant skin, even or non-blotchy texture tone and/or texture, removal or reduction of the appearance of such features as wrinkles or fine lines, surface roughness, folds or sagging (such as on the tissues of the cheeks, jowels, or brow), surface vessels, age spots/pigmentation, redness, scars from acne or other sources, and reduction of pore size and appearance.

In order mitigate the effects of aging on skin at least one skin treatment is provided. By “providing a skin treatment,” it is meant initiating exposure of an expanse of skin to light, terminating exposure to the light; and applying a benefit agent to the expanse of skin after a delay following the termination of the exposure to the light. Thus a “skin treatment” includes a light treatment followed by at least one topical treatment. More detail regarding exposing the skin to light and administering a benefit agent is provided below.

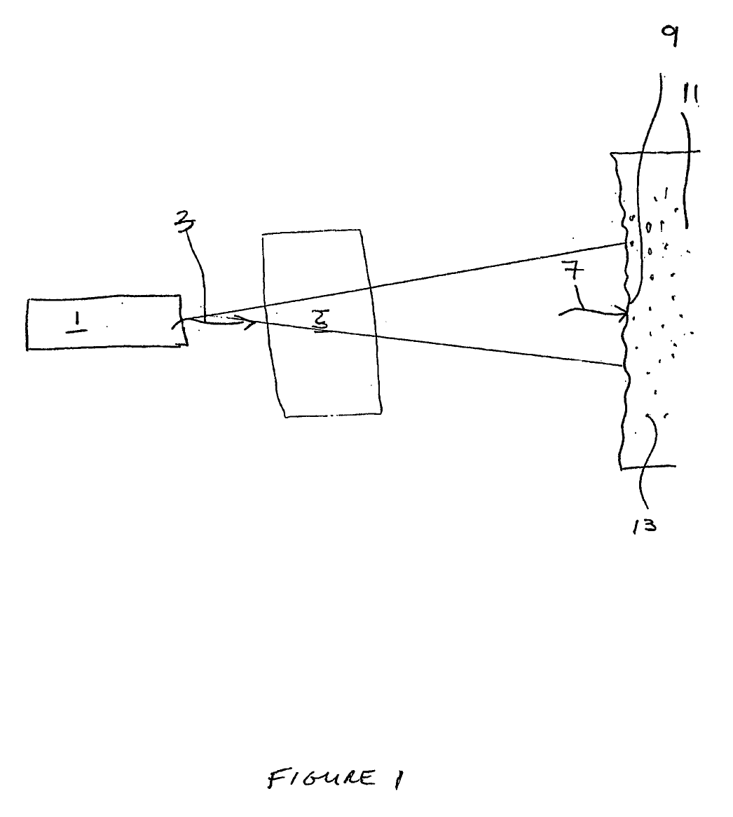

Referring to FIG. 1, a light source 1 provides light. Generally, the light source 1 is a pulsed or continuous wave source that emits an emitted light 3. The emitted light 3 may be spectrally concentrated or spectrally diffuse (i.e., broadband). The emitted light 3 may be subsequently filtered, attenuated, amplified, polarized, or otherwise modified by one or more optical elements 5 before it reaches an expanse of skin 11 to which it is directed. At the point which the light reaches an outer surface 9 of the expanse of skin 11 interacts with the skin, the light consists of an incident light 7.

The incident light 7 includes an “active portion” that is within a spectral range from about 400 nanometers (nm) to about 850 nm. Note that the emitted light and/or the incident light may or may not also include wavelengths outside of this active portion, but emission outside of the active portion is not required.

In one embodiment of the invention, the incident light is primarily within the 400 nanometers (nm) to about 850 nm spectral range. By “primarily within” it is meant that 80% or more of the total energy of the incident light is within the 400 nm to 850 nm. In one embodiment of the invention, the incident light 7 is substantially within the spectral range of about 400 nm to about 850 nm. By “substantially within the spectral range of about 400 nm to about 850 nm,” it is meant that 90% or more of the total energy is within the 400 nm to 850 nm. In another embodiment of the invention, incident light 7 is substantially free of ultraviolet radiation (i.e., less than about 1% of the total energy of the incident light 7 is in the spectral range from about 200 nm to about 400 nm).

The active portion is generally capable of being absorbed by one or more types of endogenous chromophores 13 present within the expanse of skin 11. The chromophores 13 include one or more of the following compounds: melanin, hemoglobin, deoxyhemoglobin, and water.

In one embodiment of the invention, the incident light 7 is primarily within one or more of the following spectral bands: (A) about 400 nm to about 500 nm; (B) about 575 nm to about 625 nm; and (C) about 600 nm to about 850 nm. These spectral bands are selected based upon the ability of various endogenous chromophores to absorb light within these bands. The incident light 7 may be primarily within one or more of these bands, any two of these bands, or primarily within all of these bands, in order to facilitate absorption by the endogenous chromophores.

The incident light 7 may include one or more spectral bands for facilitating particular anti-aging effects. For example, the incident light 7 may be primarily within about 400 nm to about 850 nm in order to reduce pigment contrast of the expanse of skin 11. In one particular embodiment the incident light is primarily within about 400 nm to about 800 nm, such as within about 580 nm to about 850 nm, such as within about 650 nm to about 850 nm to facilitate absorption that is primarily by melanin (as opposed to hemoglobin, deoxyhemoglobin, or water).

In another embodiment of the invention, the incident light 7 may be primarily within about 575 nm to about 625 nm in order to stimulate collagen and reduce wrinkles or fine lines on the expanse of skin 11. Light within the range of about 575 nm to about 625 nm reduces fine line and wrinkles and stimulates collagen by heating blood contained in the dermal vasculature, inducing one or more wound healing responses, i.e., blood may become a radiator of heat to the surrounding tissue.

In another embodiment of the invention, the incident light 7 maybe primarily within about 600 nm to about 750 nm in order to reduce inflammation or redness on the expanse of skin 11. Without wishing to be bound by theory, such incident light 7 reduces inflammation and redness by being absorbed by both melanin and hemoglobin.

In general, by concentrating the wavelengths of the incident light 7 to those below 850 nm, the primary endogenous chromophores that absorb the incident light 7 are melanin, hemoglobin, deoxyhemoglobin, rather than water. As such, it is possible to use incident light with lower energy density than if one were concentrating the wavelengths of the incident light 7 in the infrared. This is beneficial in that ablation of the epidermis (which may cause permanent scarring of the skin and/or pain) is prevented.

Furthermore, while the incident light 7 is generally not energetic enough to ablate the epidermis, the incident light 7 impinges upon the expanse of skin 11 with an energy density that is generally sufficient to provide localized thermal heating (such as to, for example, raise the temperature of the skin by less than about 10 Celsius degrees) and a beneficial wound-healing response.

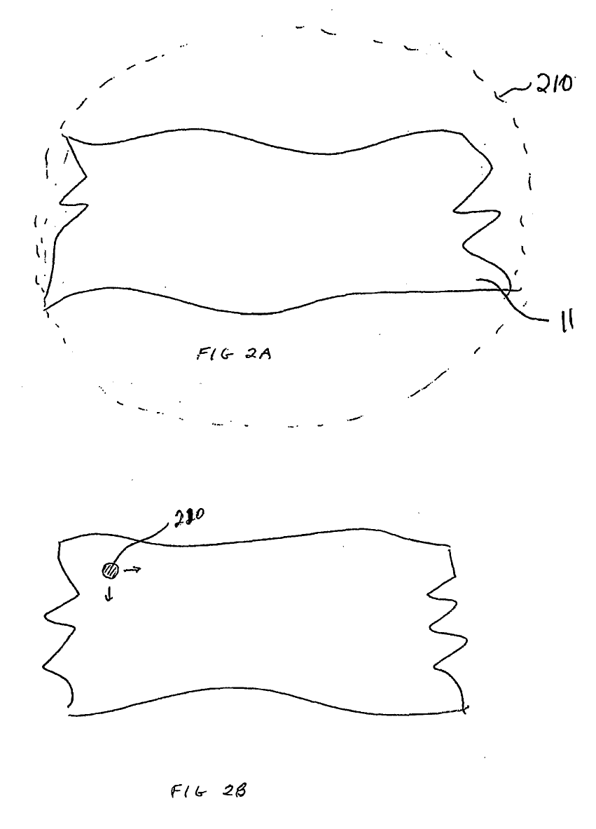

The energy density of the incident light 7 is generally within a range of about 5 J/cm2 and about 100 J/cm2, such as between about 5 J/cm2 and about 50 J/cm2. By “energy density of the incident light” 7, it is meant the energy of the incident light 7 divided by the area of a spot 210, as shown in FIG. 2A, over which the energy extends, the area determined as it impinges upon the outer surface 9 of the expanse of skin 11. Note that the terms “energy density” and “fluence” are used interchangeably throughout this disclosure. The spot 210 may have an area of about 0.5 cm² to about 10 cm². Area of spot 210 is also referred to as “spot size” in this specification.

The energy density of the incident light 7 is delivered over a particular time that may be, for example in a range of about 1 millisecond (msec) to about 60 minutes. Note that shorter times are generally more suitable for higher fluence, and longer times are more suitable for lower fluence.

The incident light 7 or the active portion thereof generally impinges upon the expanse of skin 11 with an irradiance that is in a range from about 1 milliwatt per square centimeter (mW/cm²) to about 100,000 watts per square centimeter (W/cm²). “Irradiance” of the incident light, is the energy density of the incident light 7 delivered to the expanse of skin 11 per unit time period.

The spot 210 may, in one embodiment of the invention, as shown in FIG. 2A, fully encompass the expanse of skin 11 to be treated. In this embodiment, there is no need for the incident light to be progressively repositioned (e.g., moved laterally across the expanse of skin 11) in order to deliver energy to the expanse of skin 11 across its entirety. Alternatively, as shown in FIG. 2B, the incident light 7 may have a spot 210 that is relatively small in area, e.g., less than about 1 cm², and may be progressively repositioned (e.g., stamped) across the expanse of skin 11 in order to treat the entire expanse of skin 11.

The incident light 7, or active portion thereof has a bandwidth. The bandwidth is determined by finding a wavelength (i.e., a maxima) within the active portion that is of maximum intensity, dividing this intensity in half (a “half max”) and locating a nearest first wavelength in one spectral direction that is incident at that half max intensity. A nearest second wavelength in the other spectral direction is then located. The difference between the first wavelength and the second wavelength is calculated as the bandwidth. Note that if multiple maxima are incident on the expanse of skin 11, then the maxima of greatest intensity is chosen to calculate the bandwidth.

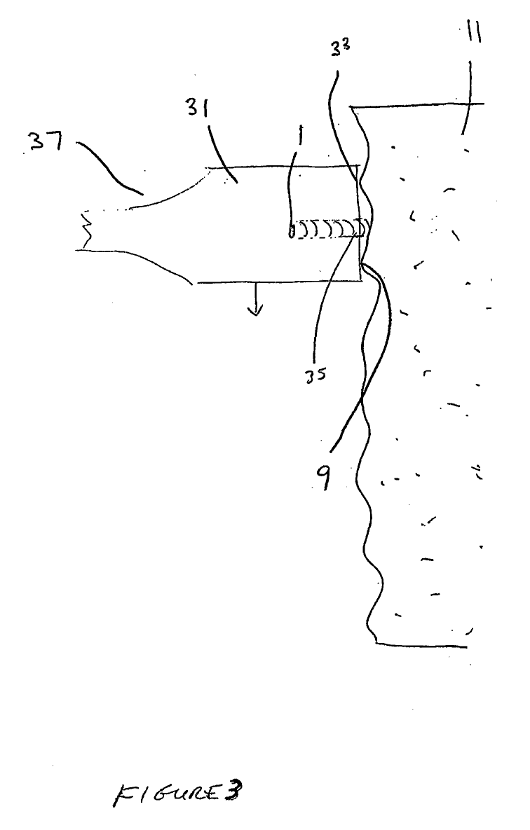

Note that while FIG. 1 depicts the light source 1 as separated from the expanse of skin 11, the distance of separation need not be great. In one embodiment of the invention, as shown in FIG. 3, the light source 1 is a part of a device 37 that includes light source 1 within a housing 31. The housing 31 (e.g., a plastic shell or container) has at least one outer surface, such as a skin-facing surface 33 that may be placed against the outer surface 9 of the expanse of skin 11, such that the light is directed through an optical window 35 to contact the expanse of skin 11. The device may, for example, be held in a user's hand and the incident light 7 may be progressively repositioned across all or portions of the expanse of skin 11. In this embodiment of the invention, the light source 1 may be maintained, for example, a distance of from about 0.5 centimeters (cm) and about 50 cm such as from about 5 cm to about 10 cm from the expanse of skin 11 during operation.

### Light Source

The light source 1 suitable for the present invention may provide, for example, a directed beam that is capable of impinging upon the expanse of skin 11 with a relatively small spot size. One suitable light source for generating a narrow spot size is a laser, such as, for example, a semiconductor laser (i.e., a “laser diode”), a ruby laser, or an Nd:YAG laser, an argon laser, a KTP laser, a dye laser, an alexandrite laser or, other lasers that may be capable of emitting light that includes the active region of wavelengths. The laser may emit light in continuous or pulsed fashion. Furthermore, suitable lasers typically have an emitted light 3 with a bandwidth less than about 2 nm. Examples of specific laser light sources that may be used in accordance with the embodiments of the present invention include those described in Altshuler (U.S. Pat. No. 6,273,884) and Anderson (U.S. patent application 20020099094A1), paragraphs 47-49. These disclosures are hereby incorporated by reference.

In another embodiment of the invention, the light source 1 may be a broadband source such as, for example a flashlamp, such as may include an incandescent, fluorescent, or chemiluminescent source. Note that specific examples of particularly suitable light sources are discussed below. Note also that the source 1 may be a broadband source that includes a filament (e.g., a tungsten filament).

Flashlamp

One notable example of a light source that may be used for practicing embodiments of the invention described herein is a pulsed, broadband source is a flashlamp (e.g. a xenon flashlamp). The flashlamp is a gas filled discharge device that takes incident electrical energy, and generates a high voltage electrical pulse that discharges the flashlamp, thereby producing pulses of electromagnetic radiation that fall within a spectral range, such as from about 200 nm to about 2000 nm. The spectral range may be adjusted by selecting a particular fill gas, a particular gas pressure, and a particular current density. Furthermore selection of a particular glass enclosure, or using one or more filters or fluorescent materials may be used to focus the incident energy within a spectral range that is narrower than the spectral range of the emitted electromagnetic radiation.

A flashlamp is suitable for providing benefits to the skin in that it emits emitted light 3 that generally extends widely (in a spatial sense) from the flashlamp, and is therefore capable of simultaneously treating an expanse of skin 11 having a large area. The area over which the light from the flashlamp extends may, however, be limited, such as by using reflectors to concentrate the light spatially.

The active portion may have a bandwidth that is greater than about 20 nm.

In one embodiment of the invention, the active portion has a bandwidth greater than about 100 nm. The incident light 7 from the flashlamp is generally non-collimated (i.e., the light is emitted in rays that are generally parallel with one another) and non-coherent (the light is emitted in rays that are not phase synchronized with one another). The flashlamp may provide pulses of light that have a duration in a range from about 1 millisecond (msec) to about several hundred milliseconds.

The flashlamp may deliver the particular range of intensity and bandwidth of the active portion that is specified above when the source 1 is placed a distance of, for example, between about 5 cm to about 10 cm (for example, when the outer surface 33 is placed in contact with the surface 9 of the expanse of skin 11).

Incident light 7 of the flashlamp may be high intensity, i.e., the active portion may deliver an energy density that is from about 10 J/cm² to about 100 J/cm². The use of high intensity flashlamp may be may be particularly suitable for use by a skilled user (e.g., a dermatologist, a medical technician, or the like). Alternatively, a high intensity flashlamp may be used for a consumer product if appropriate safety features are employed (e.g., such as those to limit over-treatment to the skin or exposure to the eye). In fact, by having a consumer use a light source having a fluence from about 10 J/cm² to about 100 J/cm², and using methods consistent with embodiments of the invention described herein, the consumer may self-treat with “at home” treatments that are highly efficacious. At home use of such devices allows for more frequent treatments than might be otherwise possible if an appointment to a professional's office were required for each treatment. More frequent treatments, even at lower dose levels provide opportunity for greater compliance and treatment efficacy. A suitable high intensity flashlamp is described in Ekhouse (U.S. Pat. No. 5,405,368), incorporated herein by reference.

Alternatively, the incident light 7 of the flashlamp maybe low intensity, i.e., the active portion may have an energy density in a range from about 5 J/cm² to about 10 J/cm². The use of low intensity radiation may be particularly suitable for use by a consumer that may not have any special or professional training in the use of the flashlamp. In general, a suitable low intensity flashlamp will have, for example, a smaller capacitor or a lower voltage than a comparable high intensity flashlamp.

Furthermore, other low intensity sources such as light emitting diodes, filament sources, fluorescent sources, and even chemiluminescent sources can provide skin benefits when used over longer exposure periods (seconds to many minutes) and with more frequent treatments than is typically used in a professional setting.

Light Emitting Diode

Another notable source for practicing embodiments of the present invention is a light emitting diode (LED). The LED is constructed from materials known in the art (e.g., compound semiconductor materials). In one embodiment of the invention, the emitted light 3 from the LED is within (A) about 400 nm to about 500 nm; (B) about 580 nm to about 600 nm; and (C) about 600 nm to about 800 nm. The narrowband source may have an emitted energy density within the active range that is greater than about 0.1 J/cm².

Referring again to FIG. 2B, the emitted light 3 from the LED may be collimated such that it impinges upon the expanse of skin 11 with spot 210 having an area less than about 10 cm². By using a source such as an LED, it is possible to provide an incident energy density that is substantially lower than that of a laser (e.g., laser diode). Radiant intensities of these LEDs may be in the range of about 1 mW/cm² to 10 mW/cm².

As shown in FIG. 3, the LED may be part of a unit such as portable unit having a exposure window across which the light is delivered such that it may contact the expanse of skin 11. The unit, and therefore the light, may be moved along or across the expanse of skin 1 to be treated in order to deliver energy thereto.

The incident light 7 from the narrowband source may be “continuous wave,” (as described in Altschuler U.S. Pat. No. 6,280,473. The disclosure of which is herein incorporated by reference). By continuous wave it is meant that the source is adapted to provide a steady-state, uninterrupted beam such that an intensity of the incident light is relatively constant over any time period less than about 1 second.

Note that while the light source 1 is described in this embodiment of the invention as “an LED,” the light source may actually include multiple LEDs in order to enhance the energy density that the light source 1 is capable of delivering.

### Benefit Agents and Compositions

Benefit agents of the present invention are generally passive in that they are substantially non-absorptive or otherwise substantially non-interactive with light within the active region. In other words, the benefit agents of the present invention are not necessarily selected in order to absorb incident light from the light source 1 in order to convert the incident light 7 to thermal energy and dissipate the thermal energy to the expanse of skin 11.

In fact, for embodiments of the invention in which the treatment is cyclical (i.e., a second treatment is provided following a first treatment). It is, to a degree, beneficial that the benefit agent not absorb the incident light 7 to a significant degree. This is because, if benefit agent remains on the expanse of skin 11 when the expanse of skin 11 is treated with light, losses due to absorption by the benefit agent either reduce the ability of the incident light 7 to provide thermal heating to the expanse of skin 11, and/or require sources of greater power (thus requiring more space, more expense, more cooling of the source, or more expense). In one embodiment of the invention, the benefit agent has an absorbance that is no greater than 0.3 Absorbance Units for any wavelength comprising the incident light 7. This absorbance can be determined through spectrophotometric measurements of a thin film of the agent applied to transparent medium, standard in the sunscreen industry, at approximately 2 mg/cm2

The benefit agent may fall into one or more classes. In one embodiment the benefit agent is an anti-wrinkle treatment. Examples of suitable anti-wrinkle treatments include, for example, retinoids such as retinol, retinyl palmitate, retinyl propionate, retinaldahyde, retinoic acid, adapelene, tazarotene, 13 cis retinoic acid, peptides that stimulate collagen synthesis such as copper-containing peptides, and Pal-KTTP sugars such as melibiose, lactose, galactose. Particularly noteworthy anti- wrinkle benefit agents are retinol and retinoic acid.

In another embodiment of the invention, the benefit agent is a keratolytic agent. Examples of suitable keratolytic agents include hydroxyacids such as alpha-hydroxyacids (AHAs), beta-hydroxyacids BHAs, and polyhyrdoxyacids. Suitable hydroxyacids include: glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucoheptonic acid, glucoheptono 1,4-lactone, gluconic acid, gluconolactone, glucuronic acid, glucuronolactone, glycolic acid, isopropyl pyruvate, methyl pyruvate, mucic acid, pyruvic acid, saccharic acid, saccaric acid 1,4-lactone, tartaric acid, and tartronic acid; beta hydroxy acids such as salicylic acid, beta-hydroxybutyric acid, beta-phenyl-lactic acid, beta-phenylpyravic acid, azeleic acid; Another useful class of keratolytics are keratolytic enzymes papain, bromaline, pepsin, trypsin.

In one embodiment the benefit agent is an anti-inflammatory/anti-redness agent. Suitable anti-inflammatory/anti-redness agents include: feverfew; alkanolamines such as ethylaminoethanol, methylaminoethanol, dimethylaminoethanolamine (DMAE), isopropanolamine, triethanolamine, isopropanoldimethylamine, ethylethanolamine, 2-butanolamine, choline and serine, catacholamines; hydrocortisone, salicylates, β sitosterol, allantoin, oat extracts, dexamethasone, caffeic acid, ginko bilboa, Stearyl glycyrrhetinate, CM Glucam, green tea extract, hyluronic acid, horsechestnut extract, licorice extract, colloidal oatmeal, tetrahydrozaline, and indomethacin. Alkanolamines such as DMAE, Feverfew, and hydrocortisone are particularly noteworthy anti-inflammatory/anti-redness agents.

In another embodiment of the invention, the benefit agent is an anti-spot/pigmentation agent. Suitable anti-spot/pigmentation agents include: depigmentation agents such as hydroquinone, catechol and its derivatives, ascorbic acid and its derivatives, kojic acid, licorice extract, azelaic acid, stearyl glycyrrhetinate, soy extracts, yohimbine, black tea extracts, and mixtures thereof; kinetin. Soy extracts are particularly noteworthy anti-spot/pigmentation agents.

In another embodiment of the invention, the benefit agent is a collagen-stimulating agent, such as peptides including Pal-KTTP, Biopeptide EL™, Biopeptide CL ™,; and copper-containing peptides such as copper polypeptide and copper peptide (GHK). Copper-containing peptides are particularly noteworthy collagen-stimulating agents.

In another embodiment of the invention, the benefit agent operates through a preventative mechanism. Suitable agents that operate through a preventative mechanism include the sub-class of anti-oxidants (for preventing oxidation) and the sub-class of sunscreens (for preventing photodamage). Suitable anti-oxidants include: Vitamin C, Vitamin E, tocopherols (alpha, gamma, delta), genistein, tocotrienols, ubiquinones, resveratrol, CoQ-10, lipoic acid, lactoferrin, pycnogenol, lycopene, magnesium ascorbyl phosphate, sodium ascorbyl phospate, isoascorbic acid, pantothenic acid, erythorbic acid, green tea extract, N acetyl cysteine, olive leaf extract, ferulic acid, feverfew, polyphenols, linoleic acid, grape seed extract, L-carnitine, lipoic acid, dihydrolipoic acid. Suitable sunscreens include: benzophenones, bomelone, butyl cinnamidopropyl trimethyl ammonium chloride, disodium distyrylbiphenyl disulfonate, potassium methoxycinnamate, butyl methoxydibenzoylmethane, octyl methoxycinnamate, oxybenzone, octocrylene, octyl salicylate, butyl salicylate, homosalate, phenylbenzimidazole sulfonic acid, ethyl hydroxypropyl aminobenzoate, menthyl anthranilate, aminobenzoic acid, cinoxate, diethanolamine methoxycinnamate,isoamyl methoxycinnamate, methyl benzylidine camphor, glyceryl aminobenzoate, titanium dioxide, zinc oxide, octotriazole, Padimate O, red petrolatum, avobenzone, Mexoryl™, Tinosorb S™ and Tinosorb M™, and mixtures thereof Particularly noteworthy sunscreens include benzophenones, butyl methoxydibenzoylmethane, octyl methoxycinnamate, oxybenzone, octocrylene, octyl salicylate, phenylbenzimidazole sulfonic acid, octotriazole, butyl dibenzoylmethane, and Tinosorb S™ and Tinosorb M™.

The benefit agent may be combined or compounded with various other auxiliary ingredients into a topical personal care composition (e.g., a cream, emulsion, serum, solution, or the like). The selection of the auxiliary ingredients may vary depending upon, for example, the ability of the benefit agent to penetrate through the skin, the specific benefit agent chosen, the particular benefit desired, the sensitivity of the user to the benefit agent, the health condition, age, and skin condition of the user, and the like. Suitable auxiliary agents include fillers, emollients and spreading agents, skin conditioners, emulsifiers, wetting agents, chelating agents, fragrances, thickeners, dyes, sensates, and the like. In one embodiment of the invention, the auxiliary ingredients have a low absorbance with respect to the incident light 7 (such as less than about 0.3 Absorbance Units, as discussed above for the benefit agent).

The benefit agent is used in a “safe and effective amount,” which is an amount that is high enough to deliver a desired skin, hair or nail benefit or to modify a certain condition to be treated, but is low enough to avoid serious side effects, at a reasonable risk to benefit ratio within the scope of sound medical judgment. Unless otherwise expressed herein, typically the benefit agent is present in the personal care composition in an amount, based upon the total weight of the composition/system, from about 0.01 percent to about 20 percent, such as from about 0.01 percent to about 5 percent (e.g., from about 0.01 percent to about 1 percent).

### Skin Treatment

In one embodiment of the invention, the expanse of skin 11 to be treated is provided a first skin treatment. The first skin treatment includes exposing the expanse of skin 11 to light primarily within the spectral range of about 400 nm to about 850 nm, said light source delivering from about 5 Joules per square centimeter to about 100 Joules per square centimeter to the skin. The light may be a source of continuous or pulsed light. In the case of pulsed light, the light treatment is terminated by terminating a series of pulses. After a period that is less than about 1 hour, exposure to the light is terminated. Note that depending upon the fluence of the light, the light may be terminated in a shorter period of time such as within a few minutes, a few seconds or even within less than one second.

Within a first delay period of less than about 24 hours after terminating the exposure to the light, a benefit agent is topically applied. By combining post-treating the expanse of skin 11 with a benefit agent after light treatment within a first delay period of 24 hours or less, a higher order of benefits is provided (i.e., a higher degree of effectiveness and/or a faster onset of benefits is provided as compared with conventional treatments). Without wishing to be bound by theory, it is believed that the inventive treatment regimen operates by multiple biological pathways (e.g., collagen formation and redness reduction). As such, the magnitude or speed of onset of benefits is not limited by the saturation of a single (i.e., light only or topical only) pathway. In order to further enhance the efficacy of the first skin treatment, the first delay may be less than 12 hours, less than 1 hour, such as from about 1 minute to about 1 hour. In particular, it is believed that by reducing the first delay period to such lower levels, a high degree of synergy is obtained between the light treatment and the topical treatment.

After a second delay period, a second skin treatment is optionally provided to the expanse of skin 11. The second skin treatment includes exposing the expanse of skin to light, terminating the exposure of the expanse of skin 11 to the light, followed by topically administering benefit agent. The second skin treatment may, for example, be similar or identical to the first skin treatment. Note that the second delay period is the time elapsed between the application of the benefit agent in the first skin treatment and the initiation of exposure of the expanse of skin 11 to light in the second skin treatment. The second delay period may be of greater duration than the first delay period. Preferably, the second delay period has a greater duration than the first delay period, more preferably a significantly greater duration. Thus, the application of the benefit agent is a post-exposure treatment, not a pre-treatment.

Note that benefit agent maybe topically applied one or more times to the expanse of skin 11 during the second delay. The benefit agent topically applied during the second delay may be the same benefit agent or same class of benefit agent applied in the first treatment, or it may be a different benefit agent or a different class of benefit agent. Topical treatments of the benefit agent may be repeated multiple times and on multiple days between light treatments. Topical and light treatments may be administered at home using a handheld light source.

The light and the topically applied benefit agent may be directed to similar benefits (e.g., collagen-stimulating/wrinkle-reducing light followed by an anti-wrinkle or collagen-stimulating benefit agent; pigment contrast-reducing light followed by anti-pigmentation agent; redness and inflammation-reducing light followed by an anti-inflammatory benefit agent). Because the topical operates through a chemical-biological pathway (the chemistry of the topical directly induces a biological response), and the light operates through a optical-biological pathway (photons of light induce a thermal response, and, in turn, a biological response), the topical and light can act synergistically and achieve a higher order of benefits.

While it is contemplated that the light and the topically applied benefit agent may be directed to similar skin care benefits, this is not required. In one embodiment of the invention the particular topical treatment and particular spectral distribution of light are chosen to complement one another and/or to act on separate, distinct pathways. Examples are provided in the paragraphs below.

For example, the light treatment may have a spectral distribution that is primarily within about 400 nm to about 850 nm in order to reduce pigment contrast. The post-treatment with benefit agent, complementary to this light treatment, may be one or more of: an anti-wrinkle treatment; a keratolytic agent; an anti-inflammatory/anti-redness agent; and a collagen-stimulating agent; or a preventative.

In another embodiment of the invention, the light treatment may have a spectral distribution that is primarily within about 550 nm to about 650 nm in order to stimulate collagen and reduce wrinkles or fine lines. The post-treatment with benefit agent, complementary to this light treatment may be one or more of: an anti-spot/pigmentation agent; an anti-inflammatory/anti-redness agent; a keratolytic agent; or a preventative.

In another embodiment of the invention, the light treatment may have a spectral distribution that is primarily within about 600 nm to about 750 nm in order to reduce inflammation or redness. The post-treatment with benefit agent, complementary to this light treatment may be one or more of: an anti-spot/pigmentation agent; an anti- wrinkle agent; a collagen-stimulating agent; a keratolytic agent; or a preventative.

### Product and Package

For convenience to the end user, one or more of light sources 1 and one or more benefit agents may be contained within an outer package and sold as a product. The product may further include instructions that indicate to the user that the user should illuminate the skin with the light source 1 and topically apply the benefit agent. The instructions may further indicate that the light source 1 and the benefit agent are to be used together (i.e., applying the benefit agent to the expanse of skin 11 after exposing the expanse of skin 11 to the light source and within about 24 hours), consistent with embodiments of the invention described herein. Note that the product may include a plurality of light sources 1 and/or benefit agents (i.e., one or more light sources 1 and/or one or more benefit agents). These light sources 1 and benefit agents may be, for example, housed in a primary package (e.g., a tube, ajar, a plastic wrap or film, and the like) that is within the outer package.

Embodiments of the invention overcome one or more drawbacks of the prior art by combining the benefits associated with a treatment based on light (i.e., wound repair) with a topical post-treatment that enhances the efficacy of the light treatment. By employing such a therapy subsequent to the light therapy, it is possible to overcome the limitations of the biological response of “light only” or “topical only” therapy by, for example, stimulating a second pathway resulting in faster onset of benefits and a higher magnitude of benefits. By combining moderate fluence of light that primarily within certain range of wavelengths with topical benefit agents, device design flexibility is enhanced since an complex cooling system is not needed, and since the treatments can be repeated more often than if professional visits might allow, treatment is highly efficacious as well as safe to use at home. Post treatment application of the topical benefit agents prevents any potential degradation of the active ingredients that may occur during the light exposures, either from direct energy absorption and degradation, or from thermal breakdown from exposures. Since the topicals are not applied before the light exposures, there is virtually unlimited time for absorption into the skin for the benefit delivery and a “wait” time between topical treatment and light exposure as is taught by others is not needed.

The following is a description of examples for treating the skin consistent with embodiments of the invention described herein. Other methods of the present invention may be performed in an analogous manner by a person of ordinary skill in the art.

### EXAMPLES

### Example 1

An expanse of skin is treated with a light from a flashlamp light source (such as one having a xenon-filled quartz-envelope and) including any necessary filters to provide a spectral distribution that is primarily within 400 to 800 nm, a bandwidth of 400 nm, a fluence of 20 to 80 J/cm² and delivered in a pulse of less than

1 second, impinges with a spot size of about 5 to 10 cm² on an expanse of skin. The light source is repositioned (stamped) across adjacent sites to complete treatment over the entire expanse of skin (e.g., an entire face). Within a first time interval of about an hour after the light treatment is completed, a benefit agent comprising retinol and an alpha-hydroxyacid such as glycolic acid is topically applied to the expanse of skin. After about 24 to 48 hours, the above steps light treatment, then topical treatment after 1 hour) are repeated. The preceding method is suitable to treat, for example, age spots and undesired pigmentation contrast present on the expanse of skin.

### Example 2

An expanse of skin is treated with a light from flashlamp light source such as one having including any necessary filters to provide a spectral distribution that is primarily within 400 to 800 nm, a bandwidth of 400 nm, a fluence of 20 to 80 J/cm² and delivered in a pulse of less than 1 second, impinges with a spot size of about 5 to 10 cm² on an expanse of skin. The light source is repositioned (stamped) across adjacent sites to complete treatment over the entire expanse of skin. Within a first time interval of about an hour after the light treatment is completed, a benefit agent comprising natural soy extracts is topically applied to the expanse of skin. After about 24 to 48 hours, the above steps (light treatment, then topical treatment after 1 hour) are repeated. The preceding method is suitable to treat, for example, age spots and undesired pigmentation contrast present on the expanse of skin.

### Example 3

An expanse of skin is treated with a light from a flashlamp light source having a spectral distribution of 575 to 625 nm, a bandwidth of less than 50 nm, a fluence of 20 to 80 J/cm² and delivered in a pulse of less than 1 second, impinges with a spot size of about 5 to 10 cm on an expanse of skin. The light source is repositioned (stamped) across adjacent sites to complete treatment over the entire expanse of skin. Within a first time interval of about an hour after the light treatment is completed, a benefit agent comprising retinol and/or a alpha hydroxy acid as glycolic acid is topically applied to the expanse of skin. After about 24 to 48 hours, the above steps (light treatment, then topical treatment after 1 hour) are repeated. The preceding method is suitable, for example, to reduce the appearance of wrinkles present on the expanse of skin.

### Example 4

An expanse of skin is treated with a light from a flashlamp light source having a spectral distribution of 575 to 625 nm, a bandwidth of 50 nm, a fluence of 20 to 80 J/cm² and delivered in a pulse of less than 1 second, impinges with a spot size of about 5 to 10 cm² on an expanse of skin. The light source is repositioned (scanned) across adjacent sites to complete treatment over the entire expanse of skin. Within a first time interval of about an hour after the light treatment is completed, a benefit agent comprising a copper-containing peptide is topically applied to the expanse of skin. After about 24 to 48 hours, the above steps (light treatment, then topical treatment after 1 hour) are repeated. The preceding method is suitable, for example, to reduce the appearance of wrinkles present on the expanse of skin.

### Example 5

An expanse of skin is treated with a light from a flashlamp light source having a spectral distribution of 575 to 625 nm, a bandwidth of 50 nm, a fluence of 20 to 80 J/cm² and delivered in a pulse of less than 1 second, impinges with a spot size of about 5 to 10 cm² on an expanse of skin. The light source is repositioned (stamped) across adjacent sites to complete treatment over the entire expanse of skin. Within a first time interval of about an hour after the light treatment is completed, a benefit agent comprising DMAE and/or, retinol, and/or an alpha hydroxyl acid such as glycolic acid is topically applied to the expanse of skin. After about 24 to 48 hours, the above steps (light treatment, then topical treatment after 1 hour) are repeated. The preceding method is suitable, for example, to reduce the appearance of wrinkles and sagging present on the expanse of skin.

### Example 6

An expanse of skin is treated with a light from a flashlamp light source having a spectral distribution of 625 to 800 nm, a bandwidth of 175 nm, a fluence of 20 to 80 J/cm² and delivered in a pulse of less than 1 second, impinges with a spot size of about 5 to 10 cm² on an expanse of skin. The light source is repositioned (stamped) across adjacent sites to complete treatment over the entire expanse of skin. Within a first time interval of about an hour after the light treatment is completed, a benefit agent comprising a natural soy extract, or a depigmentation such as hydroquinone or retinol is topically applied to the expanse of skin. After about 24 to 48 hours, the above steps (light treatment, then topical treatment after 1 hour) are repeated. The preceding method is suitable, for example, to even skin tone across the expanse of skin.

### Example 7

An expanse of skin is treated with a light from a flashlamp light source having a spectral distribution of 575 to 625 nm, a bandwidth of 50 nm, a fluence of 20 to 80 J/cm² and delivered in a pulse of less than 1 second, impinges with a spot size of about 5 to 10 cm on an expanse of skin. The light source is repositioned (stamped) across adjacent sites to complete treatment over the entire expanse of skin. Within a first time interval of about an hour after the light treatment is completed, a benefit agent comprising a retinol or and/or a alpha hydroxy acid as glycolic acid is topically applied to the expanse of skin. After about 24 to 48 hours, the above steps (light treatment, then topical treatment after 1 hour) are repeated. The preceding method is suitable, for example, to reduce the appearance of rough skin texture present on the expanse of skin.

### Example 8

An expanse of skin is treated with a light from a flashlamp light source having a spectral distribution of 625 to 700 nm, a bandwidth of 75 nm, a fluence of 5 to 50 J/cm² and delivered in a pulse of less than 1 second, impinges with a spot size of 500 cm² on an expanse of skin. Within a first time interval of about an hour after the light treatment is completed, a benefit agent comprising DMAE or an anti-inflammatory agent such as fever-few extract or natural soy extract is topically applied to the expanse of skin. After about 24 to 48 hours, the above steps (light treatment, then topical treatment after 1 hour) are repeated. The preceding method is suitable, for example, to reduce redness on the expanse of skin.

### Example 9

An expanse of skin is treated with a light from a flashlamp light source having a spectral distribution of 575 to 625 nm, a bandwidth of 50 nm, a fluence of 20 to 80 J/cm² and delivered in a pulse of less than 1 second, impinges with a spot size of about 5 to 10 cm² on an expanse of skin. The light source is repositioned (stamped) across adjacent sites to complete treatment over the entire expanse of skin. Within a first time interval of about an hour after the light treatment is completed, a benefit agent comprising DMAE or an anti-inflammatory such as fever few extract or natural soy extract is topically applied to the expanse of skin. After about 24 to 48 hours, the above steps (light treatment, then topical treatment after 1 hour) are repeated. The preceding method is suitable, for example, to reduce redness present on the expanse of skin.

### Example 10

An expanse of skin is treated with a light from a flashlamp light source having a spectral distribution of 400 to 800 nm, a bandwidth of 400 nm, a fluence of 5 to 10 J/cm² and delivered in a time period of less than 30 minutes, impinges with a spot size of 400 to 500 cm² on an expanse of skin. Within a first time interval of about an hour after the light treatment is completed, a benefit agent comprising retinol and an alpha-hyrdoxyacid such as glycolic acid is topically applied to the expanse of skin. After about 24 to 48 hours, the above steps (light treatment, then topical treatment after 1 hour) are repeated. The preceding method is suitable, for example, to reduce age spots present on the expanse of skin.

### Example 11

An expanse of skin is treated with a light from a filament light source having a spectral distribution of 575 to 625 nm, a bandwidth of 50 nm, a fluence of 5 to 10 J/cm² and delivered in a time period of less than 30 minutes, impinges with a spot size of 400 to 500 cm² on an expanse of skin. Within a first time interval of about an hour after the light treatment is completed, a benefit agent comprising retinol and an alpha-hyrdoxyacid such as glycolic acid is topically applied to the expanse of skin. After about 24 to 48 hours, the above steps (light treatment, then topical treatment after 1 hour) are repeated. Alternatively, the benefit agent may include a natural soy extract, with or without retinol. The preceding method is suitable, for example, to reduce age spots present on the expanse of skin.

### Example 12

An expanse of skin is treated with a light from a light emitting diode source (e.g., one that includes compound semiconductor-based thin films) having a spectral distribution of 575 to 625 nm, a bandwidth of 50 nm, a fluence of 5 to 10 J/cm² and delivered in a time period of less than 30 minutes, impinges with a spot size of 400 to 500 cm² on an expanse of skin. Within a first time interval of about an hour after the light treatment is completed, a benefit agent comprising retinol and an alpha-hyrdoxyacid such as glycolic acid is topically applied to the expanse of skin. After about 24 to 48 hours, the above steps (light treatment, then topical treatment after 1 hour) are repeated. The preceding method is suitable, for example, to reduce the appearance of wrinkles present on the expanse of skin.

### Example 13

An expanse of skin is treated with a light from a filament light source having a spectral distribution of 400 to 800 nm, a bandwidth of 400 nm, a fluence of 5 to 10 J/cm² and delivered in a time period of less than 30 minutes, impinges with a spot size of 400 to 500 cm² on an expanse of skin. Within a first time interval of about an hour after the light treatment is completed, a benefit agent comprising retinol and an alpha-hyrdoxyacid such as glycolic acid is topically applied to the expanse of skin. After about 24 to 48 hours, the above steps (light treatment, then topical treatment after 1 hour) are repeated. The preceding method is suitable, for example, to reduce the appearance of wrinkles present on the expanse of sin.

### Example 14

An expanse of skin is treated with a light from either (a) a filament light source or (b) a plurality of LEDs having a collectively broad emission spectrum. The spectra distribution of the light is 400 to 800 nm, a bandwidth of 400 nm, a fluence of 5 to 10 J/cm² and delivered in a time period of less than 30 minutes, impinges with a spot size of 400 to 500 cm on an expanse of skin. Within a first time interval of about an hour after the light treatment is completed, a benefit agent comprising DMAE and an alpha-hyrdoxyacid such as glycolic acid is topically applied to the expanse of skin. After about 24 to 48 hours, the above steps (light treatment, then topical treatment after 1 hour) are repeated. The preceding method is suitable, for example, to reduce sagging present on the expanse of skin. The light treatment may be performed by a skilled user, in a clinical setting, such as a dermatologist's office, or by a lay user, such as in a household.

### Example 15

An expanse of skin is treated with a light from a filament light source having a spectral distribution of 400 to 800 nm, a bandwidth of 400 nm, a fluence of 5 to 10 J/cm² and delivered in a time period of less than 30 minutes, impinges with a spot size of 400 to 500 cm² on an expanse of skin. Within a first time interval of about an hour after the light treatment is completed, a benefit agent comprising a sunscreen such as octyl methoxycinnamate, oxybenzone, or butyl dibenzoylmethane is topically applied to the expanse of skin. After about 24 to 48 hours, the above steps (light treatment, then topical treatment after 1 hour) are repeated. The preceding method is suitable, for example, to reduce sagging present on the expanse of skin as well as protecting the skin from ultraviolet radiation between treatments.

While the foregoing is directed to various embodiments of the invention, other and further embodiments may be devised without departing from the basic scope thereof.

What is claimed is:

1. A method of mitigating effects of aging on skin comprising the steps of:
a. providing a first skin treatment to an expanse of skin comprising;
i. exposing an expanse of skin to light having a wavelength of about 400 nm to about 850 nm and a fluence of about 5 J/cm2 to about 100 J/cm2;
ii. terminating the exposure of the skin to the light; and
iii. applying a first benefit agent to the expanse of skin after a delay following the termination;
wherein the light is suitable to perform a function selected from the group consisting of exciting porphyrins associated with the expanse of skin into an energetic state suitable for destroying acne-causing micro-organisms, heating lipids present in sebaceous glands within the expanse of skin in order to modulate the flow of sebum in said sebaceous glands, reducing inflammation, and combinations of these functions; and after a delay
b. providing a second skin treatment to the same expanse of skin comprising;.
i. initiating exposure of an expanse of skin to light;
ii. terminating the exposure of the expanse of skin to the light after a period, and
iii. applying the first benefit agent treatment to the expanse of skin after a delay following the termination.

2. A method of mitigating effects of aging on skin comprising the steps of:
a. exposing an expanse of skin to light having a wavelength of about 400 nm to about 850 nm and a fluence of about 5 J/cm² to about 100 J/cm²;
b. terminating the exposure of the skin to the light; and
c. applying a benefit agent to the expanse of skin after a delay of less than about 12 hours following the termination.

3. A method of promoting a topical composition comprising the step of instructing a user to topically apply said composition to an expanse of skin affected by acne following an exposure of said expanse of skin to light, wherein the light is:
a. substantially free of ultraviolet radiation;
b. has a wavelength primarily of about 400 nm to about 850 nm; and
c. provides a fluence of about 5 J/cm to about 100 J/cm²;
d. has selected wavelengths and/or wavelength bands, primarily within a wavelength range of about 400 nm to about 850 nm; and
e. delivers from about 0.01 Watt/cm² to about 100 W/cm² to the skin wherein the total fluence delivered is less than 100 J/cm².

4. The method of any of claims 1, 2, and 3, wherein the light exposure terminates after about one hour.

5. The method of any of claims 1, 2, and 3, wherein the benefit agent comprises at least one component that is suitable to provide anti-microbial action that is complementary to either said modulating of said sebum by said band of light, or complementary to said reduction of inflammation by said light.

6. The method of any of claims 1, 2, and 3, wherein the benefit agent comprises at least one component that is suitable to provide sebum-modulating action that is complementary to either said destruction of said acne-causing microorganisms or complementary to said reduction of inflammation by said light

7. The method of any of claims 1, 2, and 3, wherein the benefit agent comprises at least one component that is suitable to provide anti-inflammation that is complementary to either said modulating of said sebum by said band of light, or complementary to said destruction of said acne-causing microorganisms.

8. The method of any of claims 1, 2, and 3, wherein the light has wavelength or band of wavelengths between about 400 nm and about 410 nm.

9. The method of any of claims 1, 2, and 3, wherein the light has wavelength or band of wavelengths between about 630 nm and about 670 nm.

10. The method of any of claims 1, 2, and 3, wherein the light has a bandwidth of less than about 20 nanometers.

11. The method of any of claims 1, 2, and 3, wherein benefit agent is selected from the group consisting of a keratolytic agent, a scar mitigator, an anti-pigmentation agent, a cleansing agent, and combinations of one or more of such agents.

12. The method of claim 11 wherein the scar mitigator comprises at least one peptide.

13. The method of claim 11 wherein the anti-pigmentation agent comprises at least one anti-spot agent.

14. The method of any of claims 1, 2, and 3, wherein benefit agent further comprises an anti-fungal agent.

15. The method of claim 1, wherein the light exposure of the second treatment terminates after about one hour.

16. The method of claim 1, wherein the delay between the first and second skin treatments is greater than the delay between terminating a light treatment and applying the first benefit agent.

17. The method of claim 1, further comprising the step of applying at least one additional benefit agent treatment during the delay between the first and second skin treatments.

18. The method of claim 3, wherein the light exposure is to be completed within 24 hours prior to said topical application.

19. A kit comprising:
a. a light source that:
i. has a wavelength primarily of about 400 nm to about 800 nm; and
ii. provides a fluence of about 5 J/cm² to about 100 J/cm²;
iii. is suitable to perform a function selected from the group consisting of exciting porphyrins associated with the expanse of skin into an energetic state suitable for destroying acne-causing micro-organisms, heating lipids present in sebaceous glands within the expanse of skin in order to modulate the flow of sebum in said sebaceous glands, reducing inflammation, and combinations of these functions
b. a benefit agent; and
c. instructions directing that at least one treatment of the benefit agent be applied to the skin within 24 hours immediately following exposure of skin to light from said light source.

20. The kit of claim 19, wherein the benefit agent comprises at least one component that is suitable to provide anti-microbial action that is complementary to either said modulating of said sebum by said band of light, or complementary to said reduction of inflammation by said light.

21. The kit of claim 19, wherein the benefit agent comprises at least one component that is suitable to provide sebum-modulating action that is complementary to either said destruction of said acne-causing microorganisms or complementary to said reduction of inflammation by said light

22. The kit of claim 19, wherein the benefit agent comprises at least one component that is suitable to provide anti-inflammation that is complementary to either said modulating of said sebum by said band of light, or complementary to said destruction of said acne-causing microorganisms.

23. The kit of claim 19, wherein the light has wavelength or band of wavelengths between about 400 nm and about 410 nm.

24. The kit of claim 19, wherein the light has wavelength or band of wavelengths between about 630 nm and about 670 nm.

25. The kit of claim 19, wherein the light has a bandwidth of less than about 20 nanometers.

26. The kit of claim 19, wherein benefit agent is selected from the group consisting of a keratolytic agent, a scar mitigator, an anti-pigmentation agent, a cleansing agent, and combinations of one or more of such agents.

27. The kit of claim 26, wherein the scar mitigator comprises at least one peptide.

28. The kit of claim 26, wherein the anti-pigmentation agent comprises at least one anti-spot agent.

29. The kit of claim 19, wherein benefit agent further comprises an anti-fungal agent.

30. The kit of claim 19, wherein benefit agent further comprises an anti-fungal agent.
